(19) United States
(12) Patent Application Publication
(10) Pub. No.: US 2008/0166316 A1
(43) Pub. Date: Jul. 10, 2008

(54) Composition for hands

(76) Inventors: David Mark Burnett, Providence, UT (US); Curtis Molen Anderson, Hyrum, UT (US); T. Gary Anderson, Hyrum, UT (US); Philip Ray Cowley, Hyde Park, UT (US)

Correspondence Address: KEELY SCHNEITER, P.C., 74 WEST 100 NORTH, P.O. BOX 675, LOGAN, UT, 84321, US

(21) Appl. No.: 12/075,983

(22) Filed: Mar. 14, 2008

Related U.S. Application Data
(62) Division of application No. 11/714,605, filed on Mar. 5, 2007.
(60) Provisional application No. 60/780,015, filed on Mar. 6, 2006.

(51) Int. Cl. A61K 31/74; A61K 8/73

(52) U.S. Cl. 424/78.02; 424/70.13; 424/70.12

(57) ABSTRACT

A composition for use on the skin includes a carbomer, a starch, vitamins, moisturizers, an astringent, a Pluronic, dimethicone, and an aluminum compound. The composition may further include water, glycerin, emulsifiers, and surfactants. The composition provides therapeutic, conditioning, and other benefits to the skin, particularly when used on the hands, and is compatible with the use of gloves, including latex gloves.

### CROSS-REFERENCE TO RELATED APPLICATIONS

This application is a divisional application claiming priority to co-pending U.S. patent application Ser. No. 11/714,605 filed Mar. 5, 2007 and entitled “Composition For Hands,” which claims priority to U.S. Provisional Patent Application No. 60/780,015 filed Mar. 6, 2006 and entitled “Antiperspirant Composition.” Each of these applications is hereby incorporated by reference in its entirety.

### BACKGROUND OF THE INVENTION

1. The Field of the Invention

The present invention relates generally to compositions for use on the hands, feet, and other parts of the body. More specifically, embodiments of the present invention relate to lotions, gels, and creams and methods of making lotions, gels, and creams for use on the hands, feet, and other parts of the body.

2. Related Technology

A wide variety of cosmetic, hygienic, and medicinal products are available for use on the skin for a wide range of purposes. Among these, products which moisturize, protect, and cleanse the skin are extremely popular. In short, topical skin products are available for alleviating and ameliorating a variety of skin and other conditions.

Not only are skin products available in a variety of forms, skin products are also available for a variety of uses. For example, skin products may be used for moisturizing the skin, cleansing the skin, and protecting the skin from harmful rays of the sun. Further, skin products have been created to cure, alleviate, or ameliorate a variety of skin conditions. For example, skin products which reduce acne, smooth wrinkles, and heal rashes are widely available.

Often, a lotion, cream, or related composition is formulated to perform more than one function when applied to the skin. For example, a body lotion may perform the function of moisturizing the skin while at the same time provide a pleasing scent to the skin. Likewise, an antibacterial or antimicrobial lotion may serve the function of moisturizing the skin while at the same time killing germs. In addition, a face lotion may moisturize the skin while at the same time providing a sunscreen to the face. By combining compositions having different functions and creating lotions and creams exhibiting a variety of characteristics manufacturers create an ever increasing number of skin care products.

While lotions, creams, and gels are able to provide a wide variety of benefits and serve many purposes when applied to the skin of various parts of the body, it is difficult to find lotions or creams that provide a wide variety of therapeutic and conditioning benefits to hands, particularly when the hands are gloved. Gloves often induce moisture build-up on the hands beneath the gloves, which interferes with the absorption of many lotions and creams. Moreover, the moisture build-up caused by wearing gloves can damage hands, providing a greater need for a therapeutic skin product to be used on the hands. Furthermore, many skin products contain substances that damage gloves. For example, petroleum based skin care products cause damage to latex gloves. In addition, the antimicrobial characteristics of soaps and solutions can be compromised by some lotions and creams.

What is needed is a composition for use on the skin, particularly hands, that provides benefits to the skin even when the skin is under a glove. Further, a composition is needed that will not damage certain types of gloves and that will not inhibit the effectiveness of antimicrobial skin products.

### BRIEF SUMMARY OF THE INVENTION

Embodiments of the present invention include compositions for use on the skin, particularly the hands, and processes for making compositions for use on the skin. A carbomer is combined with a starch, moisturizers, and vitamins, forming a mixture. An astringent is combined with a Pluronic mixture, which includes an aluminum compound mixed with an alcohol. The astringent and Pluronic mixture is further combined with the carbomer mixture, thus forming a composition for the skin. The composition is therapeutic and conditioning for the skin, may include antibiotic and antimicrobial properties, and dries quickly. Further, the composition is compatible with glove use, even latex gloves. In some embodiments of the invention the benefits derived from use of the composition on the skin are enhanced by use of the composition with gloves.

These and other aspects of the present invention will become more fully apparent from the following description and appended claims, or may be learned by the practice of the invention as set forth hereinafter.

### BRIEF DESCRIPTION OF THE DRAWINGS

To further clarify the above and other aspects of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The drawings are not drawn to scale. The invention will be described and explained with additional specificity and detail through the use of the accompanying drawings in which:

FIG. 1 is a block diagram of the components of a composition for use on the skin;

FIG. 2 is a chart showing percentages of components of a composition for use on the skin; and,

FIG. 3 is a block diagram showing a process for making a composition for use on the skin.

### DETAILED DESCRIPTION OF EXEMPLARY EMBODIMENTS

Embodiments of the present invention include compositions for application on the hands, feet, and other areas of the body to provide therapeutic, conditioning, and other benefits to the skin. A skin protectant, such as dimethicone, is combined with vitamins, moisturizers, astringents, starch, and other ingredients to create a composition for use on the skin. The composition of the present invention is characterized by a blend of therapeutic, conditioning, re-hydrating, and other properties. In addition, the composition not only provides therapeutic and moisture control benefits to the skin, it is also compatible with chlorhexadinegluconate (“CHG”) and can be used while wearing gloves.

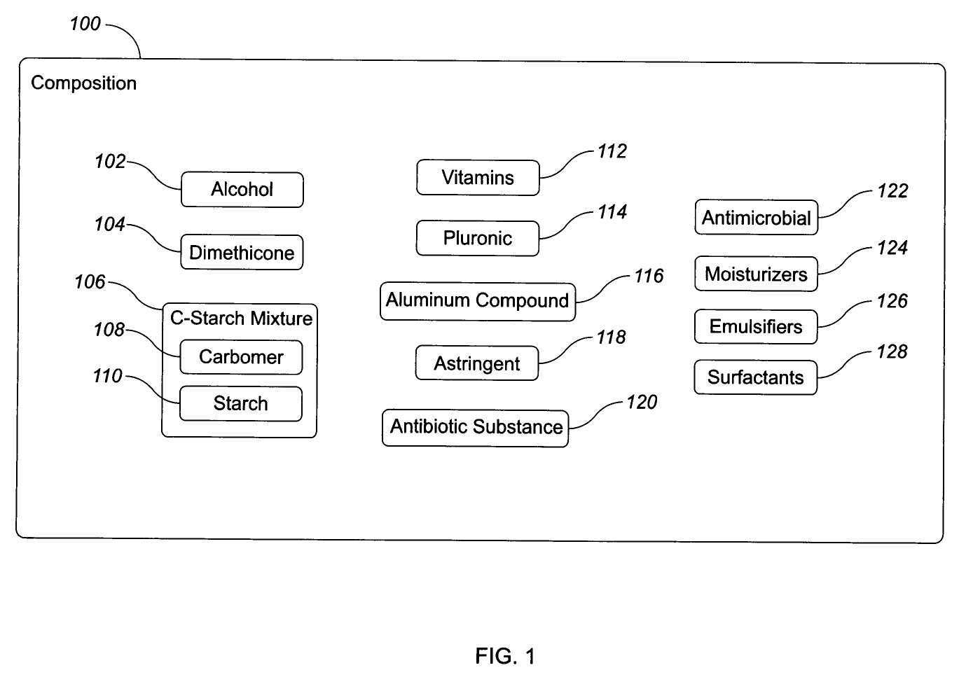

With attention now to FIG. 1, the composition 100 of the present invention includes alcohol 102, dimethicone 104, and a C-starch mixture 106, which includes a carbomer 108 and a starch 110. For purposes of this description, the term mixture is defined as a combination or blend of different elements and does not connote any specific technical meaning such as may be used in chemistry. C-starch mixture 106 may also be referred to as a hydrogel matrix. In one embodiment of the invention, the carbomer used in the C-starch mixture 106 is carbomer 940. In addition, in one embodiment of the invention the starch used in the C-starch mixture 106 is a solanum tuberosum starch, also known as a potato starch. In one embodiment of the invention a starch is included in the C-starch mixture 106 and is also included as an additional component of the composition 100 that is not a part of the C-starch mixture. Further, in one embodiment of the invention the composition also includes vitamins 112, such as, for example, vitamin E, dexpanthenol, or any other vitamin.

In addition to the above listed ingredients, composition 100 also includes a Pluronic 114 and an aluminum compound 116, such as, for example, aluminum acetate. Also, the composition of the present invention includes an astringent 118. In one embodiment of the invention the astringent 118 is witch hazel.

Moreover, in one embodiment of the invention the composition further includes one or more antibiotic substances 120 for treating skin conditions. Also, embodiments of the invention may include an antimicrobial substance 122, such as, for example, chlorhexadinegluconate, or, any other antimicrobial substance. Composition 100, in one embodiment of the invention, further includes moisturizers 124 and emulsifiers 126. In one embodiment of the invention moisturizers 124 include, for example, isopropyl palmitate. Further, in one embodiment of the invention emulsifiers 126 include, for example, lecithin, sorbitan, and polysorbate. Finally, composition 100 may include surfactants 128, which, in one embodiment of the invention, may include, for example, poloxamer. It is to be appreciated that other ingredients not listed above or shown in FIG. 1 may be present in composition 100. Such ingredients may include, for example, distilled water, glycerin, and aloe.

When the components of the composition 100 are combined, a composition is formed which is therapeutic and conditioning for the skin and is compatible for use with gloves, including, for example, latex gloves. Moreover, when used with gloves the composition of the present invention controls under-glove moisture, provides barrier protection, dries quickly, and functions to re-hydrate the skin.

More particularly, C-starch mixture 106 provides a barrier protection to hands when the composition 100 is in use. The C-starch mixture 106 is characterized by lipophobic and lipophilic properties that prevent oils, chemicals, surfactants, or other materials or substances that may be harmful to the skin, from contacting or attaching to the skin. The substances listed above instead attach to an outer barrier of the C-starch mixture 106. In other words, the substances listed above bond with or attach to the carbomer 108 and not the skin of the person using the composition 100. Thus, when the composition is washed off of the skin, any oils, chemicals, surfactants, or other harmful materials or substances are washed away from the skin with the C-starch mixture 106 of the composition 100.

In addition to providing a barrier protection to the skin, the C-starch mixture 106 also re-hydrates the skin. The C-starch mixture 106 functions to absorb or take in water, such as sweat, that is released from the skin. The water is added into or combined with the C-starch mixture 106, which is in contact with the skin. When the skin is surrounded by a glove, the C-starch mixture 106, with the added moisture from the skin, is kept in contact with the skin and functions to re-hydrate the skin. In other words, the glove may act as a bandage holding the C-starch mixture 106 against the skin. Thus, the C-starch mixture 106 forms a coating or web over the skin that facilitates regeneration of skin cells and re-hydrates and the skin.

While a glove used with the composition 100 may increase benefits provided by the composition 100 to the skin, the composition 100 will not compromise the integrity of the glove material or cause the glove to disintegrate or degenerate. For example, the composition 100 will not harm or affect latex gloves. Further, if the skin has been treated with CHG or other antimicrobial products prior to application of the composition 100, the beneficial effects of the CHG or other antimicrobial products will not be compromised by the components of the composition 100.

Moreover, when the composition 100 is used under a glove the composition 100 controls moisture that may be caused by the presence of the glove over the skin. The aluminum compound 116 functions to remove excess water from the skin. The excess water may then be absorbed by the C-starch mixture 106.

Excess moisture present on the skin may further be reduced by the aluminum compound 116, which is one embodiment of the invention is aluminum acetate. The aluminum compound 116 may act as an antiperspirant, thus helping reduce moisture produced by the hand when the hand is gloved. The antiperspirant effects of aluminum compounds, such as aluminum compound 116, are particularly helpful in embodiments of the present invention because wearing gloves may oftentimes enhance perspiration of the hands. In one embodiment of the invention the Pluronic 114 functions as a delivery mechanism to deliver the aluminum compound to the skin, thus helping to curtail perspiration on the hands.

Other benefits and effects of the composition 100 include conditioning and protecting. Vitamins 112 included in composition 100 work to provide nutrients to the skin and to condition the skin. Further, in one embodiment of the invention aloe 124 is also included in composition 100 and further provides conditioning benefits to the skin. The skin is further protected by dimethicone 104. Finally, the composition 100 dries in a short amount of time, such as, for example, less than ten seconds. Such quick drying time allows a person using the composition to put on gloves shortly after applying to composition without having to put gloves over wet skin. Also, while the composition 100 provides conditioning and protecting benefits to the skin, the composition 100 may also include antibiotic, antimicrobial, and antibacterial components to further benefit the skin. Overall, the combination of the components included in the composition 100 provides therapeutic and other beneficial effects to the skin of a person using the composition 100.

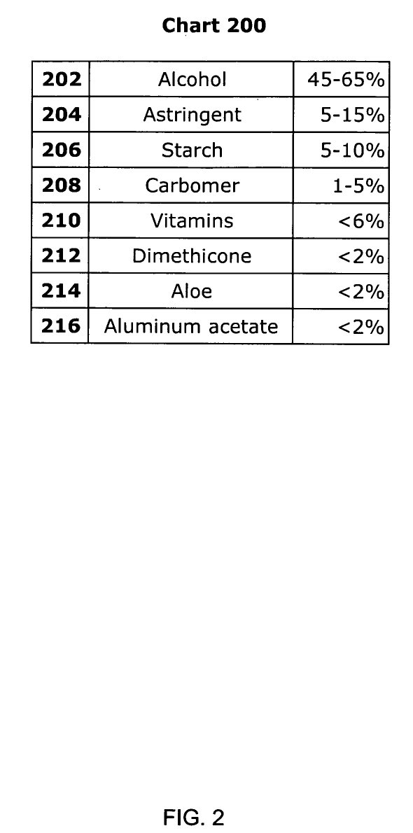

With attention now to FIG. 2, a chart 200 showing specific percentages of each of the components of the composition of the present invention used in one embodiment of the invention is shown. Beginning with the component having the highest percentage, in one embodiment of the invention the composition is 45-65% alcohol, such as, for example, ethyl alcohol and/or cetyl alcohol, as shown at line 202. In one embodiment of the invention where the percentage of ethyl alcohol is greater than 61% the composition of the present invention provides antibacterial benefits when applied to the skin.

In addition to alcohol, in one embodiment of the invention the composition includes 5-15% astringent, as shown at line 204, 5-10% starch, as shown at line 206, and 1-5% carbomer, as shown at line 208. In addition, in one embodiment of the invention the composition is less than 6% vitamins, as shown at line 210, and less than 2% each of dimethicone, aloe, and aluminum acetate, shown at lines 212, 214, and 216, respectively. In one embodiment of the invention the composition further includes one or more of water, glycerin, moisturizers, emulsifiers, and surfactants.

Although not shown in FIG. 2, in one embodiment of the invention the composition of the present invention is about 50% ethyl alcohol and about 16% water. Further, in one embodiment of the invention the composition is about 11% witch hazel, about 7% cetyl alcohol, about 6% solanum tuberosum starch, about 2% each of carbomer and glycerin, and about 1.5% each of isopropyl palmitate and lecithin. Finally, in one embodiment of the invention the composition further includes about 1% each of dimethicone, sorbitan, and polysorbate, about 0.15% vitamin E, about 0.03% each of dexpanthenol and aloe, about 0.5% poloxamer, and about 0.3% aluminum acetate.

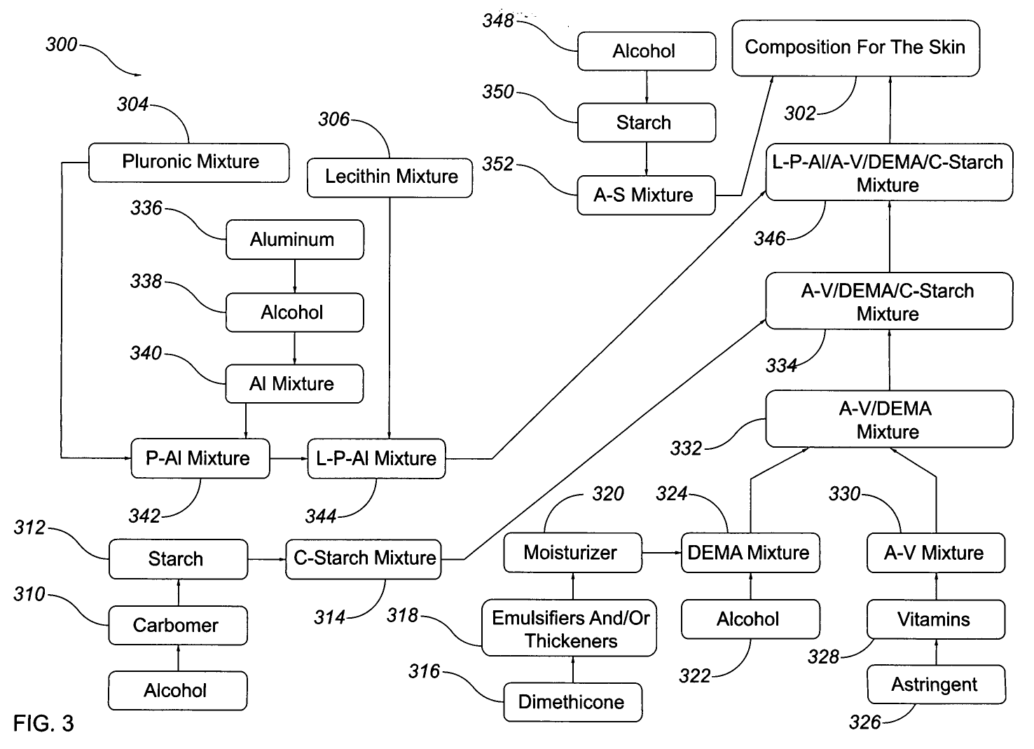

With attention now to FIG. 3, a block diagram showing a process for making the composition 300 in one embodiment of the present invention is shown. Beginning at stage 302, a Pluronic mixture is formed. In one embodiment of the invention the Pluronic mixture includes, in addition to the Pluronic, water. In one embodiment of the invention the Pluronic pre-mixture may be refrigerated before being further combined with additional components of the composition 300.

Moving now to stage 304, a lecithin mixture is shown. In one embodiment of the invention the lecithin mixture includes, in addition to the lecithin, a moisturizer, such as, for example, isopropyl palmitate. Further, in one embodiment of the invention the lecithin pre-mixture shown at stage 304 is stored at room temperature until the lecithin pre-mixture is combined with other components of the composition 300.

Directing attention now to stage 306, alcohol, such as, for example, ethyl alcohol, is combined with a carbomer, such as, for example, carbomer 940, as shown at stage 308. Next, a starch, such as, for example, potato starch, is added at stage 310 and an alcohol-carbomer-starch mixture, or C-starch mixture is formed at stage 312. In one embodiment of the invention the C-starch mixture is stored at room temperature before being combined with other components of composition 300. In addition, in one embodiment of the invention the C-starch mixture shown at stage 312 may include additional ingredients such as, for example, water.

In addition to the C-starch mixture shown at stage 312, composition 300 also includes dimethicone, as shown at stage 314, which is combined with emulsifiers and/or thickeners, as shown at stage 316. In one embodiment of the invention the emulsifers and/or thickeners include polysorbate and/or sorbitan. At stage 318 a moisturizer is added to the dimethicone and emulsifiers and/or thickeners. In one embodiment of the invention the moisturizer is isopropyl palmitate. Finally, at stage 320, alcohol, such as, for example, cetyl alcohol, is combined with the dimethicone, emulsifiers and/or thickeners, and moisturizer to form a dimethicone-emulsifier-thickener-moisturizer-alcohol mixture, also referred to as a DEMA mixture. In one embodiment of the invention the cetyl alcohol shown at stage 320 is heated before the dimethicone-emulsifier-moisturizer mixture is combined with the alcohol, as shown at stage 322. Further, in one embodiment of the invention the DEMA mixture shown at stage 322 is heated during mixing of the alcohol with the dimethicone-emulsifier-moisturizer mixture and the temperature of the resulting DEMA mixture, shown at stage 322, is maintained until the DEMA mixture is combined with other components of composition 300.

Another component of the composition 300 is an astringent, as shown at stage 324. In one embodiment of the invention the astringent is witch hazel. At stage 326 the astringent is combined with vitamins, such as, for example, vitamin E, dexpanthenol, and any other vitamin, thus forming an astringent-vitamin mixture, or A-V mixture, as shown at stage 328. In one embodiment of the invention the A-V mixture includes additional ingredients such as, for example, glycerin, aloe, and other ingredients.

When the A-V mixture is formed, as shown at stage 328, the A-V mixture may be warmed and then combined with the DEMA mixture, as shown at stage 330. After the A-V mixture and DEMA mixtures have been combined at stage 330, the resulting A-V/DEMA mixture may be further combined with the C-starch mixture, as shown at stage 332, thus forming an A-V/DEMA/C-starch mixture. In one embodiment of the invention the A-V/DEMA/C-starch mixture shown at stage 332 is continuously mixed until further components of the composition 300 can be added to the A-V/DEMA/C-starch mixture.

An additional component of composition 300 is an aluminum compound, such as aluminum acetate, as shown at stage 334. At stage 336 alcohol, such as ethyl alcohol, as added to the aluminum compound, thus forming an aluminum-alcohol mixture, or Al mixture, as shown at stage 338. At stage 340 the Pluronic mixture of stage 302 is added to the Al mixture, thus forming a Pluronic-aluminum-alcohol mixture, or P-Al mixture. Finally, at stage 342 the lecithin mixture shown at stage 304 is added to the P-Al mixture, thus forming a lecithin-Pluronic-aluminum-alcohol mixture, or L-P-Al mixture. In one embodiment of the invention the L-P-Al mixture is continuously mixed until the L-P-Al mixture is combined with other components of the composition 300. The L-P-Al mixture of stage 342 is further combined with the A-V/DEMA/C-starch mixture of stage 332, thus forming a lecithin-Pluronic-aluminum-astringent-vitamin-dimethicone-emulsifier-moisturizer-carbomer-strach mixture, or L-P-Al/A-V/DEMA/C-starch mixture, as shown at stage 344.

With attention now to stage 346, a further components of composition 300 is shown. Alcohol, such as ethyl alcohol, shown at stage 346, is combined with starch, such as potato starch, as shown at stage 348, thus forming an alcohol-starch mixture, also referred to as an A-S mixture, as shown at stage 350. Finally, the A-S mixture is combined with the L-P-Al/A-V/DEMA/C-starch mixture, thus forming a composition for the skin, as shown at stage 352. In one embodiment of the invention the A-S mixture is slowly added to the L-P-Al/A-V/DEMA/C-starch mixture to form a composition for the skin.

It is to be appreciated that the components listed herein do not form an exhaustive list of components of the composition of the present invention and other components may be added to the composition at the various stages outlined above. For example, in one embodiment of the invention the composition further includes one or more emulsifiers and one or more surfactants. Further, in one embodiment of the present invention the composition is in the form of a cream. In another embodiment of the present invention the composition is a lotion, and in a further embodiment of the present invention the composition is a gel.

Moreover, it is also to be appreciated that although one exemplary process for making the composition of the present invention is described with reference to FIG. 3, other processes for making the composition of the present invention are included in additional embodiments of the invention.

Thus, the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of claims are to be embraced within their scope.

We claim:

1. A process for making a composition for use on the skin, comprising:
forming a Pluronic mixture;
forming a lecithin mixture;
mixing a first alcohol with a carbomer and a first starch, thus forming a alcohol-carbomer-starch mixture;
mixing dimethicone with one or more emulsifiers and one or more moisturizers, thus forming a dimethicone-emulsifier-moisturixer mixture;
adding a second alcohol to the dimethicone-emulsifier-moisturizer mixture, thus forming a dimethicone-emulsifier-moisturizer-alcohol mixture;
mixing an astringent with vitamins to form an astringent-vitamin mixture;
combining the astringent-vitamin mixture with the dimethicone-emulsifier-moisturizer-alcohol mixture, thus forming an astringent-vitamin-dimethicone-emulsifier-moisturizer-alcohol mixture;
adding the alcohol-carbomer-starch mixture to the astringent-vitamin-=dimethicone-emulsifier-moisturizer-alcohol mixture, thus forming an alcohol-carbomer-starch-astringent-vitamin-dimethicone-emulsifier-moisturizer-alcohol mixture;
mixing an aluminum compound with a third alcohol, thus forming an aluminum compound mixture;
adding the Pluronic mixture to the aluminum compound mixture;
adding the lecithin mixture to the aluminum compound mixture;
combining the aluminum compound mixture with the alcohol-carbomer-starch-astringent-vitamin-dimethicone-emulsifier-moisturizer-alcohol mixture, thus forming an aluminum compound-alcohol-carbomer-starch-astringent-vitamin-dimethicone-emulsifier-moisturizer-alcohol mixture;
mixing a fourth alcohol with a second starch, thus forming an alcohol-starch mixture; and,
combining the alcohol-starch mixture with the aluminum compound-alcohol-carbomer-starch-astringent-vitamin-dimethicone-emulsifier-moisturizer-alcohol mixture, thus forming an alcohol-starch-aluminum compound-alcohol-carbomer-starch-astringent-vitamin-dimethicone-emulsifier-moisturizer-alcohol mixture.

2. The process as recited in claim 1, wherein the Pluronic mixture comprises one or more of: a Pluronic; and, water.

3. The process as recited in claim 1, wherein the lecithin mixture comprises one or more of: lecithin; and, isopropyl palmitate.

4. The process as recited in claim 1, wherein the first alcohol, second alcohol, third alcohol, and fourth alcohol comprise one or more of: ethyl alcohol; and, cetyl alcohol.

5. The process as recited in claim 1, wherein the carbomer is carbomer 940.

6. The process as recited in claim 1, wherein the first starch and the second starch comprise potato starch.

7. The process as recited in claim 1, wherein the one or more emulsifiers comprises one or more of: polysorbate 80; and, sorbitan monooleate.

8. The process as recited in claim 1, wherein the one or more moisturizers is isopropyl palmitate.

9. The process as recited in claim 1, wherein the astringent is witch hazel.

10. The process as recited in claim 1, wherein the vitamins comprises one or more of: vitamin E; and, dexpanthenol.

11. The process as recited in claim 1, wherein the astringent-vitamin mixture further comprises one or more of: aloe; and, glycerin.

12. The process as recited in claim 1, wherein the aluminum compound is aluminum acetate.

13. The process as recited in claim 1, further comprising adding one or more surfactants.

14. The process as recited in claim 13, wherein the one or more surfactants is poloxamer 407.
